SEQUENCE LISTING (8 sequences)

SEQ ID NO: 1 - DNA, 15 bp, Artificial Sequence
  Description of Artificial Sequence Synthetic PNA probe nucleobase sequence
tcacacaatcgtaac

SEQ ID NO: 2 - DNA, 15 bp, Artificial Sequence
  Description of Artificial Sequence Synthetic PNA probe nucleobase sequence
cctcgaatgtgctat

SEQ ID NO: 3 - DNA, 15 bp, Artificial Sequence
  Description of Artificial Sequence Synthetic PNA probe nucleobase sequence
cccagctagcgtgcc

SEQ ID NO: 4 - DNA, 15 bp, Artificial Sequence
  Description of Artificial Sequence Synthetic PNA probe nucleobase sequence
cccagctcgcgtgcc

SEQ ID NO: 5 - DNA, 15 bp, Artificial Sequence
  Description of Artificial Sequence Synthetic PNA blocker probe nucleobase sequence
tcacgcaaacgtaac

SEQ ID NO: 6 - DNA, 15 bp, Artificial Sequence
  Description of Artificial Sequence Synthetic PNA blocker probe nucleobase sequence
cctcgaatgcgctat

SEQ ID NO: 7 - DNA, 15 bp, Artificial Sequence
  Description of Artificial Sequence Synthetic PNA probe nucleobase sequence
cctctgatgggtagg

SEQ ID NO: 8 - DNA, 15 bp, Artificial Sequence
  Description of Artificial Sequence Synthetic PNA probe nucleobase sequence
ccttctgatgggcag

(19) United States
(12) Patent Application Publication
(10) Pub. No.: US 2005/0272078 A1
(43) Pub. Date: Dec. 8, 2005

(54) Peptic nucleic acid probes for analysis of Enterococcus faecium

(75) Inventors: Mark Fiandaca, Princeton, MA (US); Henrik Stender, Gentofte (DK)

Correspondence Address: EDWARDS & ANGELL, LLP, P.O. BOX 55874, BOSTON, MA, 02205, US

(73) Assignee: AdvanDx, Inc.

(21) Appl. No.: 11/117,642

(22) Filed: Apr. 27, 2005

Related U.S. Application Data
(63) Continuation-in-part of application No. PCT/US03/34350, filed on Oct. 27, 2003.
(60) Provisional application No. 60/421,657, filed on Oct. 28, 2002.

(51) Int. Cl. C12Q 1/68; C07K 7/08; C07K 7/06

(52) U.S. Cl. 435/6; 530/328; 530/329; 530/330

(57) ABSTRACT

Disclosed are PNA based probes that include a nucleobase sequence suitable for the analysis of Enterococcus faecium. In one embodiment, the probe is complementary to a target sequence of Enterococcus faecium 23 rRNA or rDNA or its complement. The invention has a wide range of important applications including use to detect Enterococcus faecium in a biological sample.

### CROSS-REFERENCE TO RELATED APPLICATIONS

The present application is a continuation-in-part of international patent application no. PCT/US03/34350 (WO 2004/039831) as filed on Oct. 27, 2003 which application claims priority to U.S. Provisional Application No. 60/421,657 as filed on Oct. 28, 2002. The disclosures of the PCT/US03/34350 and 60/421,657 applications are each incorporated herein by reference.

### FIELD OF THE INVENTION

The present invention relates to peptide nucleic acid (PNA) probes, PNA probe sets and methods for the analysis of Enterococcus faecium optionally present in a sample. The invention further relates to diagnostic kits comprising such PNA probes.

### BACKGROUND OF THE INVENTION

The diagnosis of infectious diseases is still often based on classical microbiology methodologies, such as culture and biochemical tests for phenotypic markers, and typically takes from 1-2 days up to weeks or even months before final diagnosis is available. In the meantime, patients are often treated empirically based on preliminary test results and clinical symptoms, which often lead to an unnecessary use of antibiotics and its sequelae.

Enterococcus faecium is an important cause of nosocomial infections, such as bacteremia, urinary tract infections and catheter-related infections and is further complicated by increasing resistance to ampicillin and more importantly vancomycin, such that vancomycin-resistant Enterococcus faecium today is endemic at major hospitals worldwide. Recently, resistance to novel antibiotics, such as linezolid has also been observed.

Conventional biochemical methods for the analysis of Enterococcus faecium are slow and misidentifications are well known. Rapid and accurate methods for detection, identification and/or quantitation of Enterococcus faecium are thus important in order to ensure optimal antibiotic therapy and patient management as well as to reduce the spread of multi-resistant strains within the hospital environment.

Despite its name, Peptide Nucleic Acid (PNA) is neither a peptide nor a nucleic acid, it is not even an acid. PNA is a non-naturally occuring polyamid that can hybridize to nucleic acid (DNA and RNA) with sequence specificity (See: U.S. Pat. No. 5,539,082) and Egholm et al., Nature 365:566-568 (1993)) according to Watson-Crick base paring rules. However, whereas nucleic acids are biological materials that play a central role in the life of living species as agents of genetic transmission and expression, PNA is a recently developed totally artificial molecule, conceived in the minds of chemists and made using synthetic organic chemistry. PNA also differs structurally from nucleic acid. Although both can employ common nucleobases (A, C, G, T, and U), the backbones of these molecules are structurally diverse. The backbones of RNA and DNA are composed of repeating phosphodiester ribose and 2-deoxyribose units. In contrast, the backbones of the most common PNAs are composed of (aminoethyl)-glycine subunits. Additionally, in PNA the nucleobases are connected to the backbone by an additional methylene carbonyl moiety. PNA is therefore not an acid and therefore contains no charged acidic groups such as those present in DNA and RNA. The non-charged backbone allows PNA probes to hybridize under conditions that are destabilizing to DNA and RNA, attributes that enable PNA probes to access targets, such as highly structured rRNA and double stranded DNA, known to be inaccessible to DNA probes (See: Stephano & Hyldig-Nielsen, IBC Library Series Publication #948. International Business 5, Communication, Southborough, Mass., pp. 19-37 (1997)). PNAs are useful candidates for investigation when developing probe-based hybridization assays because they hybridize to nucleic acids with sequence specificity. PNA probes, however are not the equivalent of nucleic acid probes in structure or function.

Comparative analysis of ribosomal RNA (rRNA) sequences or genomic DNA sequences corresponding to said rRNA (rDNA) has become a widely accepted method for establishing phylogenetic relationships between bacterial species (Woese, Microbiol. Rev. 51:221-271 (1987)), and Bergey's Manual of systematic bacteriology has been revised based on rRNA or rDNA sequence comparisons. Ribosomal RNA or rDNA sequence differences between closely related species enable design of specific probes for microbial identification and thus enable diagnostic microbiology to be based on a single genetic marker rather than a series of phenotypic markers as characterizing traditional microbiology (Delong et al., Science 342:1360-1363 (1989)). In addition to providing information about species identity mutations in the Enterococcus faecium rRNA sequences have been associated with resistance to certain antibiotics, such as linezolid, where particularly G2576T point mutation, but also other mutations, such as C2610G, G2505A, C2512T and G2513T (Prystowsky et al., Antimicrob Agents Chemother. 45:2154-2156 (2001)) have been described. However, the very high degree of rRNA or rDNA sequence similarity between Enterococcus species (Patel et al., J. Clin. Microbiol. 36:3399-3407 (1998)) and between Enterococcus faecium strains with point mutations associated with resistance to antibiotics complicates the design of specific probes for analysis of Enterococcus faecium. A PNA probe for identification of Enterococcus faecalis as well as a PNA probe targeting most other Enterococcus species have recently been described, but a PNA probe for Enterococcus faecium was not described (Oliveira et al., Abstract #D-2003, Interscience Conference on Antimicrobial Agents and Chemotherapy, Sep. 27-30, 2002, San Diego, Calif.). Those two PNA probes allow Enterococcus faecium to be ruled-out or ruled-in, however, they do not provide final identification of Enterococcus faecium. Accordingly, there is a need in the field for effective PNA probes that can be used to analyze Enterococcus faecium in a wide range of biological samples.

### SUMMARY OF THE INVENTION

This invention is directed to PNA probes and their use as well as kits useful for the analysis of Enterococcus faecium optionally present in a sample of interest. In accordance with claim 1, the PNA probes are directed to 23S rRNA or the genomic sequences corresponding to said rRNA (rDNA) or its complement. In preferred embodiments the probes of this invention are used for in situ hybridization analysis of Enterococcus faecium optionally present in a sample, most preferably the in situ hybridization analysis is fluorescence in situ hybridization analysis.

In one embodiment, this invention is directed to PNA probes for detection, identification and/or quantitation of Enterococcus faecium.

In another embodiment, this invention is directed to PNA probes for the detection of single point mutations of Enterococcus faecium associated with resistance to antibiotics. The PNA probes are particularly useful for specific analysis of Enterococcus faecium without cross-hybridization to Enterococcus durans and Enterococcus hirae, the two closest related Enterococcus species.

These PNA probes have the inherent physico/chemical characteristics of PNA probes as compared to nucleic acid probes, such that rapid and accurate analysis can be performed using just a single PNA probe. Furthermore, Enterococcus faecium is a gram-positive bacterium with a relatively rigid cell wall where the improved penetration of PNA probes also offers an advantage as compared to nucleic acid probes when applied in fluorescence in situ hybridization assays. Where nucleic acid probes require fixation and permeabilization with cross-linking agents and/or enzymes (for example see Kempf et al., J. Clin. Microbiol 38:830-838 (2000)), these PNA probes can be applied directly following smear preparation as exemplified in example 1.

In a preferred embodiment, these PNA probes have a relatively short nucleobase sequence, such as 8-17 bases, most preferably they are 15 nucelobases as described in example 1. Naturally occurring nucleic acid probes are typically at least 18 nucleobases (For example see Kempf et al., J. Clin. Microbiol 38:830-838 (2000)) due to their lower Tm values. This difference provides these PNA probes with better discrimination to closely related non-target sequences with only a single or just a few nucleobase difference(s) as required for analysis of Enterococcus faecium 23S rRNA or rDNA.

PNA probe nucleobase sequences according to the invention are selected from the group consisting of: TCA CAC MT CGT MC (Seq. Id. No. 1), CCT CGA ATG TGC TAT (Seq. Id. No. 2), CCC AGC TAG CGT GCC (Seq. Id. No. 3) and CCC AGC TCG CGT GCC (Seq. Id. No. 4). One or more of these probes, or the complements thereof, are included in the most preferred probe sets of this invention.

Preferably probes of this invention are labeled with at least one detectable moiety, wherein the detectable moiety or moieties are selected from the group consisting of: a conjugate, a branched detection system, a chromophore, a fluorophore, a spin label, a radioisotope, an enzyme, a hapten, an acridinium ester and a luminescent compound. Fluorescent labeled probes of this invention may be self-reporting, most preferably self-reporting fluorescent probes of this invention are PNA Linear Beacons.

PNA probes of this invention may also be unlabeled, for instance as described in Examples 3 and 4 where unlabeled probes are used as a Blocker probes.

In other conceptions of the present invention PNA probes are included which contain moieties that add functionality to the probe. Such moieties include but are not limited to spacer and linker groups. Likewise, PNA probes of this invention encompass probes attached to a solid support such as but not limited to a membrane, a slide, an array, a bead, or a particle.

Probe sets of this invention include two or more PNA probes for the analysis of Enterococcus faecium optionally present in a sample. Probe sets are preferably labeled with a detectable moiety. Probe sets may be labeled with the same detectable moiety, or they may be differently labeled for independent analysis of probe signals. It is within the conception of this invention that two or more differently labeled fluorescent probes of a probe set may be used to create a third signal by coincidental fluorescence.

The method according to the invention comprises contacting a sample with one or more of the PNA probes described above. According to the method, the presence, absence and/or number of Enterococcus faecium organisms in the sample are then detected, identified and/or quantitated and/or the susceptibility to antibiotics is determined by correlating the hybridization, under suitable hybridization conditions, of the probing nucleobase sequence of the probe to the target sequence. Consequently, the analysis is based on a single assay with a definitive outcome. In contrast, current routine methods for analysis of Enterococcus faecium are based on multiple phenotypic characteristics involving multiple tests.

In preferred embodiments the methods of this invention are used for in situ hybridization analysis of Enterococcus faecium optionally present in a sample, most preferably the in situ hybridization analysis is fluorescence in situ hybridization analysis. In preferred methods of the invention, the sample is a biological sample, including but not limited to blood, urine, secretion, sweat, sputum, stool, mucous, or cultures thereof.

Preferred methods of the invention optionally include non-labeled blocking probes to reduce or eliminate hybridization of PNA probes to non-target sequences Methods of this invention do not include the use of cross-linking reagents or enzymes prior to hybridization.

The methods of this invention may also be used to detect nucleic acid targets generated, synthesized or amplified in a reaction. Preferred methods for generating, synthesizing or amplifying targets include PCR, LCR, SDA, TMA, RCA and Q-beta replicase.

Methods of the invention include those in which the targets are immobilized to a surface, such as a membrane, a slide, a bead, or a particle and which may furthermore be a component of an array. Optionally, the methods may include PNA probes which are immobilized to a surface such as a membrane, a slide, a bead, or a particle, and may furthermore be a component of an array.

In a highly preferred embodiment of the invention, the medical treatment of a patient includes i.) obtaining a sample from the patient, ii.) determining the presence, amount and/or identity of Enterococcus faecium in the sample, and iii.) optionally administering at least one antibiotic compound towards vancomycin resistant enterococci (VRE). Such antibiotic compounds include but are not limited to linezolid, quinupristin-dalfopristin and daptomycin.

In still another embodiment, this invention is directed to kits suitable for performing an assay that detect, identify and/or quantitate Enterococcus faecium optionally present in a sample and/or determination of antibiotic resistance. The kits of this invention comprise one or more PNA probes and other reagents or compositions that are selected to perform an assay or otherwise simplify the performance of an assay. In particularly, the combined analysis of species identity and antibiotic resistance is well-suited for optimal patient treatment as certain antibiotics are approved for vancomycin-resistant Enterococcus faecium, but not for vancomycin-resistant Enterococcus faecalis and therefore require information about both species identity and susceptibility to antibiotics. Preferred kit formats include kits designed to perform in situ hybridization assays, and kits designed to perform real-time PCR assays. Preferred kits are designed to examine samples such as clinical specimens, or cultures thereof.

Those of ordinary skill in the art will appreciate that a suitable PNA probe need not have exactly these probing nucleobase sequences to be operative but often modified according to the particular assay conditions. For example, shorter PNA probes can be prepared by truncation of the nucleobase sequence if the stability of the hybrid needs to be modified to thereby lower the Tm and/or adjust for stringency. Similarly, the nucleobase sequence may be truncated at one end and extended at the other end as long as the discriminating nucleobases remain within the sequence of the PNA probe. Such variations of the probing nucleobase sequences within the parameters described herein are considered to be embodiments of this invention.

The PNA probes, methods and kits of this invention have been demonstrated to be both sensitive and specific for Enterococcus faecium. Moreover, the assays described herein are rapid (less than 3 hours) and capable of analysis of Enterococcus faecium in a single assay.

Those of ordinary skill in the art will also appreciate that the complement probing sequence is equally suitable for assays, such as but not limited to real-time PCR, that are using rDNA as target.

### DETAILED DESCRIPTION OF THE INVENTION

I. Definitions:

a. As used herein the term “nucleobase” means those naturally occurring and those non-naturally occurring heterocyclic moieties commonly known to those who utilize nucleic acid technology or utilize peptide nucleic acid technology to thereby generate polymers that can sequence specifically bind to nucleic acids.

b. As used herein, the term “nucleobase sequence” means any segment of a polymer that comprises nucleobase-containing subunits. Non-limiting examples of suitable polymers or polymer segments include oligodeoxynucleotides, oligoribonucleotides, peptide nucleic acids, nucleic acid analogs, nucleic acid mimics, and/or chimeras.
c. As used herein, the term “target sequence” means the nucleobase sequence that is to be detected in an assay.

d. As used herein, the term “probe” means a polymer (e.g. a DNA, RNA, PNA, chimera or linked polymer) having a probing nucleobase sequence that is designed to sequence-specifically hybridize to a target sequence of a target molecule of an organism of interest.

e. As used herein, “analyze” means that the individual bacteria are marked for detection, identification and/or quantitation and/or for determination of resistance to antibiotics (antimicrobial susceptibility).
f. As used herein, the term “peptide nucleic acid” or “PNA” means any oligomer, linked polymer or chimeric oligomer, comprising two or more PNA subunits (residues), including any of the polymers referred to or claimed as peptide nucleic acids in U.S. Pat. Nos. 5,539,082, 5,527,675, 5,623,049, 5,714,331, 5,736,336, 5,773,571, 5,786,461, 5,837,459, 5,891,625, 5,972,610, 5,986,053, 6,107,470 and 5,357,163. In the most preferred embodiment, a PNA subunit consists of a naturally occurring or non-naturally occurring nucleobase attached to the aza nitrogen of the N-[2-(aminoethyl)] glycine backbone through a methylene carbonyl linkage.

g. As used herein, the terms “label” and “detectable moiety” are interchangeable and shall refer to moieties that can be attached to a probe to thereby render the probe detectable by an instrument or method.

2. Description

I. General:

PNA Synthesis:

Methods for the chemical assembly of PNAs are well known (see: U.S. Pat. Nos. 5,539,082, 5,527,675, 5,623,049, 5,714,331, 5,736,336, 5,773,571, 5,786,461, 5,837,459, 5,891,625, 5,972,610, 5,986,053 and 6,107,470).

PNA Labeling:

Preferred non-limiting methods for labeling PNAs are described in U.S. Pat. Nos. 6,110,676, 6,361,942, 6,355,421, the examples section of this specification or are otherwise well known in the art of PNA synthesis and peptide synthesis.

Labels:

Non-limiting examples of detectable moieties (labels) suitable for labeling PNA probes used in the practice of this invention would include a dextran conjugate, a branched nucleic acid detection system, a chromophore, a fluorophore, a spin label, a radioisotope, an enzyme, a hapten, an acridinium ester and a chemiluminescent compound.

Other suitable labeling reagents and preferred methods of attachment would be recognized by those of ordinary skill in the art of PNA, peptide or nucleic acid synthesis.

Preferred haptens include 5 (6)-carboxyfluorescein, 2,4-dinitrophenyl, digoxigenin, and biotin.

Preferred fluorochromes (fluorophores) include 5 (6)-carboxyfluorescein (Flu), 6-((7-amino-4-methylcoumarin-3-acetyl) amino) hexanoic acid (Cou), 5 (and 6)-carboxy-X-rhodamine (Rox), Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye Cyanine 7 (Cy7) Dye, Cyanine 9 (Cy9) Dye (Cyanine dyes 2,3,3.5,5 and 5.5 are available as NHS esters from Amersham, Arlington Heights, Ill.), JOE, Tamara or the Alexa dye series (Molecular Probes, Eugene, Oreg.).

Preferred enzymes include polymerases (e.g. Taq polymerase, Kienow PNA polymerase, T7 DNA polymerase, Sequenase, DNA polymerase 1 and phi29 polymerase), alkaline phosphatase (AP), horseradish peroxidase (HRP) and most preferably, soy bean peroxidase (SBP).

Unlabeled Probes:

The probes that are used for the practice of this invention need not be labeled with a detectable moiety to be operable within the methods of this invention, for example when attached to a solid support

Self-Indicating Probes:

Beacon probes are examples of self-indicating probes which include a donor moiety and a acceptor moiety. The donor and acceptor moieties operate such that the acceptor moieties accept energy transferred from the donor moieties or otherwise quench signal from the donor moiety. Though the previously listed fluorophores (with suitable spectral properties) might also operate as energy transfer acceptors, preferably, the acceptor moiety is a quencher moiety. Preferably, the quencher moiety is a non-fluorescent aromatic or heteroaromatic moiety. The preferred quencher moiety is 4-((-4-(dimethylamino) phenyl) azo) benzoic acid (dabcyl). In a preferred embodiment, the self-indicating Beacon probe is a PNA Linear Beacon as more fully described in U.S. Pat. No. 6,485,901.

In another embodiment, the self-indicating probes of this invention are of the type described in WIPO patent application W097/45539. These self-indicating probes differ as compared with Beacon probes primarily in that the reporter must interact with the nucleic acid to produce signal.

Spacer/Linker Moieties:

Generally, spacers are used to minimize the adverse effects that bulky labeling reagents might have on hybridization properties of probes. Preferred spacer/linker moieties for the nucleobase polymers of this invention consist of one or more aminoalkyl carboxylic acids (e.g. aminocaproic acid), the side chain of an amino acid (e.g. the side chain of lysine or ornithine), natural amino acids (e.g. glycine), aminooxyalkylacids (e.g. 8-amino-3,6-dioxaoctanoic acid), alkyl diacids (e.g. succinic acid), alkyloxy diacids (e.g. diglycolic acid) or alkyldiamines (e.g. 1,8-diamino-3,6-dioxaoctane).

Hybridization Conditions/Stringency:

Those of ordinary skill in the art of nucleic acid hybridization will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Optimal stringency for a probe/target sequence combination is often found by the well known technique of fixing several of the aforementioned stringency factors and then determining the effect of varying a single stringency factor. The same stringency factors can be modulated to thereby control the stringency of hybridization of a PNA to a nucleic acid, except that the hybridization of a PNA is fairly independent of ionic strength. Optimal stringency for an assay may be experimentally determined by examination of each stringency factor until the desired degree of discrimination is achieved.

Suitable Hybridization Conditions:

Generally, the more closely related the background causing nucleic acid sequences are to the target sequence, the more carefully stringency must be controlled. Blocking probes may also be used as a means to improve discrimination beyond the limits possible by optimization of stringency factors.

Suitable hybridization conditions will thus comprise conditions under which the desired degree of discrimination is achieved such that an assay generates an accurate (within the tolerance desired for the assay) and reproducible result.

Aided by no more than routine experimentation and the disclosure provided herein, those of skill in the art will easily be able to determine suitable hybridization conditions for performing assays utilizing the methods and compositions described herein. Suitable in-situ hybridization or PCR conditions comprise conditions suitable for performing an in-situ hybridization or PCR procedure. Thus, suitable in-situ hybridization or PCR conditions will become apparent to those of skill in the art using the disclosure provided herein, with or without additional routine experimentation.

Blocking Probes:

Blocking probes are nucleic acid or non-nucleic acid probes that can be used to suppress the binding of the probing nucleobase sequence of the probing polymer to a non-target sequence. Preferred blocking probes are PNA probes (see: U.S. Pat. No. 6,110,676). It is believed that blocking probes operate by hybridization to the non-target sequence to thereby form a more thermodynamically stable complex than is formed by hybridization between the probing nucleobase sequence and the non-target sequence. Formation of the more stable and preferred complex blocks formation of the less stable non-preferred complex between the probing nucleobase sequence and the non-target sequence. Thus, blocking probes can be used with the methods, kits and compositions of this invention to suppress the binding of the probes to a non-target sequence that might be present and interfere with the performance of the assay. Blocking probes are particularly advantageous for discrimination to the phylogenetically closest related Enterococcus durans and Enterococcus hirae.

Probing Nucleobase Sequence:

The probing nucleobase sequence of a probe of this invention is the specific sequence recognition portion of the construct. Therefore, the probing nucleobase sequence is a nucleobase sequence designed to hybridize to a specific target sequence wherein the presence, absence or amount of the target sequence can be used to directly or indirectly detect the presence, absence or number of organisms of interest in a sample. Consequently, with due consideration to the requirements of a probe for the assay format chosen, the length and sequence composition of the probing nucleobase sequence of the probe will generally be chosen such that a stable complex is formed with the target sequence under suitable hybridization conditions.

The preferred probing nucleobase sequence of the probes of this invention that are suitable for the detection, identification and/or enumeration of Enterococcus faecium comprise a nucleobase sequence of: TCA CAC MT CGT MC (Seq. Id No. 1), CCT CGA ATG TGC TAT (Seq. Id. No. 2) and the complements thereto.

The preferred probing nucleobase sequence of the probes of this invention that are suitable for the determination of antimicrobial susceptibility of Enterococcus faecium comprise a nucleobase sequence of: CCC AGC TAG CGT GCC (Seq. Id No. 3), CCC AGC TCG CGT GCC (Seq. Id. No. 4) and the complements thereto, where Seq. Id. No. 3 is for detection of mutation associated with resistance to linezolid and Seq. Id. No. 4 is for detection of the wild type.

This invention contemplates that variations in these identified probing nucleobase sequences shall also provide probes that are suitable for the analysis of Enterococcus faecium. Variation of the probing nucleobase sequences within the parameters described herein are considered to be an embodiment of this invention.

Common variations include, deletions, insertions and frame shifts. Additionally, a shorter probing nucleobase sequence can be generated by truncation of the sequence identified above.

A probe of this invention will generally have a probing nucleobase sequence that is exactly complementary to the target sequence. Alternatively, a substantially complementary probing nucleobase sequence might be used since it has been demonstrated that greater sequence discrimination can be obtained when utilizing probes wherein there exists one or more point mutations (base mismatch) between the probe and the target sequence (See: Guo et al., Nature Biotechnology 15: 331-335 (1997)). Consequently, the probing nucleobase sequence may be only 90% homologous to the probing nucleobase sequences identified above. Substantially complementary probing nucleobase sequence within the parameters described above are considered to be an embodiment of this invention.

Complements of the probing nucleobase sequence are considered to be an embodiment of this invention, since it is possible to generate a suitable probe if the target sequence to be detected has been amplified or copied to thereby generate the complement to the identified target sequence.

Detection, Identification and/or Enumeration:

By detection is meant analysis for the presence or absence of the organism optionally present in the sample. By identification is meant establishment of the identity of the organism by genus and species name. By quantitation is meant enumeration of the organisms in a sample. Some assay formats provide simultaneous detection, identification and enumeration (for example see Stender, H. et al., J. Microbiol. Methods. 45:31-39 (2001), others provide detection and identification (for example see Stender, H. et al., Int. J. Tuberc. Lung Dis. 3:830-837 (1999) and yet other assay formats just provide identification (for example see Oliveira, K et al. J. Clin. Microbiol. 40:247-251 (2002)).

Antibiotic Resistance

By determination of resistance to antibiotics is meant analysis of an organisms susceptibility to antibiotics based on specific genes or mutations associated with resistance or susceptibility to antimicrobial agents.

II. Preferred Embodiments of the Invention:

a. PNA Probes:

In one embodiment, the PNA probes of this invention are suitable for detecting, identifying and/or quantitating Enterococcus faecium or for the determination of resistance to antibiotics of Enterococcus faecium optionally present in a sample. General characteristics (e.g. length, labels, nucleobase sequences, linkers etc.) of PNA probes suitable for the analysis have been previously described herein. The preferred probing nucleobase sequence of PNA probes of this invention are listed in Table 1.

| | |
|---|---|
| Sequence ID | Nucleobase sequence |

| | |
|---|---|
| Seq. Id. No. 1 | TCA CAC AAT CGT AAC |
| Seq. Id. No. 2 | CCT CGA ATG TGC TAT |
| Seq. Id. No. 3 | CCC AGC TAG CGT GCC |
| Seq. Id. No. 4 | CCC AGC TCG CGT GCC |

The PNA probes of this invention may comprise only a probing nucleobase sequence (as previously described herein) or may comprise additional moieties. Non-limiting examples of additional moieties include detectable moieties (labels), linkers, spacers, natural or non-natural amino acids, or other subunits of PNA, DNA or RNA. Additional moieties may be functional or non-functional in an assay. Generally however, additional moieties will be selected to be functional within the design of the assay in which the PNA probe is to be used. The preferred PNA probes of this invention are labeled with one or more detectable moieties selected from the group consisting of fluorophores, enzymes and haptens.

In preferred embodiments, the probes of this invention are used in in-situ hybridization (ISH) and fluorescence in-situ hybridization (FISH) assays. Excess probe used in an ISH or FISH assay typically must be removed so that the detectable moiety of the specifically bound probe can be detected above the background signal that results from still present but unhybridized probe. Generally, the excess probe is washed away after the sample has been incubated with probe for a period of time. However, the use of self-indicating probes is a preferred embodiment of this invention, since there is no requirement that excess self-indicating probe be completely removed (washed away) from the sample since it generates little or no detectable background. In addition to ISH or FISH assays, self-indicating probes comprising the selected probing nucleobase sequence described herein are particularly useful in all kinds of homogeneous assays such as in real-time PCR or useful with self-indicating devices (e.g. lateral flow assay) or self-indicating arrays.

b. PNA Probe Sets

Probe sets of this invention comprise two of more PNA probes. In one embodiment, some of the PNA probes of the set can be blocking probes. The preferred nucleobase sequences of blocker probes are TCA CGC AAA CGT MC (Seq. Id No. 1 Block), CCT CGA ATG CGC TAT (Seq. Id. No. 2Block) or the complements thereto used together with TCA CAC MT CGT MC (Seq. Id No. 1), CCT CGA ATG TGC TAT (Seq. Id. No. 2) or the complements thereto, respectively. In other embodiments, the probe set comprises at least two PNA probes selected from TCA CAC MT CGT MC (Seq. Id No. 1), CCT CGA ATG TGC TAT (Seq. Id. No. 2), CCC AGC TAG CGT GCC (Seq. Id No. 3), and CCC AGC TCG CGT GCC (Seq. Id. No. 4) and the complements thereto.

c. Methods:

In another embodiment, this invention is directed to a method suitable for analysis of Enterococcus faecium optionally in a sample. The general and specific characteristics of PNA probes suitable for the analysis of Enterococcus faecium have been previously described herein. Preferred probing nucleobase sequences are listed in Table 1.

The method for analysis of Enterococcus faecium in a sample comprises contacting the sample with one or more PNA probes suitable for hybridization to a target sequence which is specific to Enterococcus faecium.

According to the method, Enterococcus faecium in the sample is then detected, identified and/or quantitated or its resistance to antibiotics is determined. This is made possible by correlating hybridization, under suitable hybridization conditions, of the probing nucleobase sequence of a PNA probe to the target sequence of Enterococcus faecium sought to be detected with the presence, absence or number of the Enterococcus faecium organisms in the sample. Typically, this correlation is made possible by direct or indirect detection of the probe/target sequence hybrid.

Fluorescence In Situ Hybridization and Real-Time PCR:

The PNA probes, methods, kits and compositions of this invention are particularly useful for the rapid probe-based analysis of Enterococcus faecium. In preferred embodiments, in-situ hybridization or PCR is used as the assay format for analysis of Enterococcus faecium. Most preferably, fluorescence in-situ hybridization (PNA FISH) or real-time PCR is the assay format. (Reviewed by Stender et al. J. Microbiol. Methods 48:1-17 (2002)). Preferably, smears for PNA FISH analysis are not treated with cross-linking agents or enzymes prior to hybridization.

Exemplary Assay Formats:

Exemplary methods for performing PNA FISH can be found in: Oliveira et J. Clin. Microbiol 40:247-251 (2002), Rigby et al., J. Clin. Microbiol. 40:2182-2186 (2002), Stender et al., J. Clin. Microbiol. 37:2760-2765 (1999), Perry-O'Keefe et al., J. Microbiol. Methods 47:281-292 (2001). According to one method, a smear of the sample, such as, but not limited to, a positive blood culture, is prepared on microscope slides and covered with one drop of the fluorescein-labeled PNA probe in hybridization buffer. A coverslip is placed on the smear to ensure an even coverage, and the slide is subsequently placed on a slide warmer or incubator at 55° C. for 90 minutes. Following hybridization, the coverslip is removed by submerging the slide into a pre-warmed stringent wash solution and the slide is washed for 30 minutes. The smear is finally mounted with one drop of mounting fluid, covered with a coverslip and examined by fluorescence microscopy.

Enterococcus faecium optimally present in a sample which may be analyzed with the PNA probes contained in the kits of this invention can be determined by several instruments, such as but not limited to the following examples: microscope (for example see Oliveira et al., J. Clin. Microbiol 40:247-251 (2002)), film (for example see Perry-O'Keefe et al., J. Appl. Microbiol. 90:180-189) (2001), camera and instant film (for example see Stender et al., J. Microbiol. Methods 42:245-253 (2000)), luminometer (for example see Stender et al., J. Microbiol. Methods. 46:69-75 (2001), laser scanning device (for example see Stender et al., J. Microbiol. Methods. 45: 31-39 (2001) or flow cytometer (for example see Wordon et al., Appl. Environ. Microbiol. 66:284-289 (2000)). Automated slide scanners and flow cytometers are particularly useful for rapidly quantitating the number of microorganisms present in a sample of interest.

Exemplary methods for performing real-time PCR using self-reporting PNA probes can be found in: Fiandaca et al., Abstract, Nucleic Acid-Based technologies. DNA/RNA/PNA Diagnostics, Washington, D.C., May 14-16, 2001, and Perry-O'Keefe et al., Abstract, International Conference on Emerging Infectious Diseases, Atlanta, 2002.

d. Kits:

In yet another embodiment, this invention is directed to kits suitable for performing an assay, which analyses Enterococcus faecium optionally present in a sample. The general and preferred characteristics of PNA probes suitable for the analysis of Enterococcus faecium have been previously described herein. Preferred probing nucleobase sequences are listed in Table 1. Furthermore, methods suitable for using the PNA probes to analyse Enterococcus faecium in a sample have been previously described herein.

The kits of this invention comprise one or more PNA probes and other reagents or compositions which are selected to perform an assay or otherwise simplify the performance of an assay used to analyze Enterococcus faecium in a sample.

e. Exemplary Applications For Using The Invention:

The PNA probes, methods and kits of this invention are particularly useful for the analysis of Enterococcus faecium in clinical samples, e.g. urine, blood, wounds, sputum, laryngeal swabs, gastric lavage, bronchial washings, biopsies, aspirates, expectorates as well as in food, beverages, water, pharmaceutical products, personal care products, dairy products or environmental samples and cultures thereof.

Additional invention embodiments are discussed below.

A. Identifying Patients Harboring Vancomycin Resistant Enterococci

In many hospitals worldwide, the prevalence of vancomycin resistant enterococci (VRE) among E. faecium is often very high whereas VRE is rarely seen in E. faecalis isolates. Early institution of aggressive therapy for VRE based on rapid and accurate identification of E. faecium will therefore be appropriate as compared to current treatment practices where aggressive therapy is often not instituted until antibiotic susceptibility testing results become available 2-3 days later. This is exemplified in a study of an intensive care unit where patients with bacteremia caused by VRE did not receive appropriate therapy until after the lab results became available (Ibrahim, E. H. et al. 2000. The influence of inadequate antimicrobial treatment of bloodstream infections on patient outcomes in the ICU setting. CHEST 118:146-155). The mortality rate within this patient group was 60% and even exceeded the mortality rate for bacteremia caused by methicillin-resistant S. aureus (MRSA). Rapid identification of E. faecium according to this invention therefore allows for aggressive therapy for VRE potentially leading to more favorable patient outcomes. Therapeutic decisions based on rapid identification of E. faecium before the antibiotic susceptibility results become available, are therefore also part of this invention. Preferably, the treatment decision is prescription and administration of at least one antibiotic (eg., less than about five and preferably one, two or three), such as linezolid, quinupristin-dalfopristin, daptomycin or other antibiotic with activity towards VRE. Typical patients for practice of this invention embodiment include those who have or are suspected of harboring VRE. Such patients are readily identified by health care givers by rapid identification of E. faecium.

B. Additional Detection Strategies

Three color detection: PNA probes for analysis of E. faecalis and enterococci other than E. faecalis have previously been described (WO2005018423). A surprising result came when testing these probes together with PNA probes of this invention. The fluorescent signal from the combined use of two probes for detection of E. faecium, one labeled with fluorescein (green) targeting the 23S rRNA and one labeled with Tamra (red) targeting the 16S rRNA was, surprisingly, golden yellow in color. The golden color is the result of coincidental detection of green and red fluorescence by the eye, or camera. Detection of the golden color only occurs when the signals are relatively close in strength, otherwise they are perceived as aberrations of either color, i.e. “greenish” or “reddish”. Also, and most importantly, the golden color only occurs when the fluorescent green and red signals emanate from what is perceived as the same point. Since ribosoma RNA targets are distributed throughout cells in an essentially random pattern they appear to occupy the same space as perceived at 50-500× magnification.

Careful use of probes, as described in Example 4, resulted in a three color assay using only two fluorescent labels, fluorescein and tamra. In the example, E. faecalis is detected by green fluorescence, E. faecium is detected by golden fluorescence, and E. hirae, and E. durans are detected by red fluorescence. Though the use of combinations of labels to produce multiple colors has been described previously for fluorescent microscopy, this is the first description of an Enterococcus probe set using coincidental fluorescence to produce an additional color.

Though probes labeled with fluorescein and tamra are described, it is within the concept of this invention that any combination of fluorescent labels could be used which produce a perceivable third color. Likewise, use of two or more labels to produce multiple perceivable colors is also envisioned. Potential fluorescent labels are included in the description. Coincidental fluorescence of two or more fluorescent moieties has been demonstrated to be useful in the generation of a spectrum of colors (Kool et al JACS 2003). Combination colors are made through “mixtures” of two or more fluorophores, and adjustment of their ratios. For example, a combination of two parts red and one part green produces a different color that one part red and two parts green. Though accurate discrimination of these various shades by eye may have a practical limit, it is not difficult to conceive of a device which could accurately perceive such subtle color variations.

E. faecium, E. faecalis, E. durans and E. hirae are closely related species with few differences between their rRNA sequences. The PNA probes (three labeled probes plus one blocker probe) used in Example 4 detect target regions which are nearly identical between Enterococcus species. The careful design and selection of these probes takes advantage of these 1-2 base differences between species enabling species-specific, color-indicated detection. Three-color multiplex analysis of E. faecium, E. faecalis and other enterococci is described here for the first time. Simultaneous identification and differentiation of E. faecalis, E. faecium and other enterococci (other than E. faecalis and E. faecium) using rapid probe based analysis is therefore within the embodiment of this invention.

Further Definitions:

Coincidental fluorescence: As used herein, the term “coincidental fluorescence” is used to describe the perception of a color which is generated by the simultaneous detection of light emissions of two or more labels located near enough in space so as to be irresolvable. The detection of coincidental fluorescence can be either by eye or a photon-sensitive device.

Detectable and Independently Detectable Moieties/Multiplex Analysis:

A multiplex hybridization assay can be performed in accordance with this invention.

In a multiplex assay, numerous conditions of interest can be simultaneously examined.

Multiplex analysis relies on the ability to sort sample components or the data associated therewith, during or after the assay is completed. In preferred embodiments of the invention, one or more distinct independently detectable moieties can be used to label two or more different probes used in an assay. The ability to differentiate between and/or quantitate each of the independently detectable moieties provides the means to multiplex a hybridization assay. Correlation of the hybridization of each of the distinctly (independently) labeled probes to particular nucleic acid sequences is indicative of presence, absence or quantity of each organism sought to be detected in the sample.

Consequently, the multiplex assays of this invention can be used to simultaneously detect the presence, absence or quantity of two or more different organisms (e.g. species of enterococci) in the same sample and in the same assay. For example, a multiplex assay may utilize two or more PNA probes, each being labeled with an independently detectable fluorophore, or a set of independently detectable fluorophores.

Accordingly, the invention provides for a method to treat a patient which in embodiment includes at least one of and preferably all of the following steps:
a) obtaining a biological sample from the patient
b) determining the presence, amount and/or identity of E. faecium; and
c) administering at least one antibiotic with activity towards vancomycin resistant enterococci (VRE). In one embodiment, the antibiotic is linezolid, quinupristin-dalfopristin, or daptomycin. The patient can have or be suspected of harboring a vancomycin resistant enterococci (VRE).

The invention further provides for a PNA probe set that includes at least one of the PNA probes provided herein, preferably two or more probes, wherein the probes to make a third color by coincidental fluorescence.

Although two or a more PNA probes will be suitable for most applications in which coincidental fluorescence detection is desired, it will often be useful to use at least one of and preferably all of the following probes: CCTCGAATGTGCTAT (Seq. Id. No. 2), CCTCGAATGCGCTAT (Seq. Id. No.2Block), CCTCTGATGGGTAGG and CCTTCTGATGGGCAG. Examples of these and other suitable probes can be found in WO2005018423, for instance.

Having described the preferred embodiments of the invention, it will now become apparent to one of skill in the art that other embodiments incorporating the concepts described herein may be used. It is felt, therefore, that these embodiments should not be limited to disclosed embodiments but rather should be limited only by the spirit and scope of the following claims.

### EXAMPLES

This invention is now illustrated by the following example, which are not intended to be limiting in any way.

### Example 1

### Analysis of Enterococcus faecium by Fluorescence In Situ Hybridization

PNA Probe Sequence

(Seq. Id. No.1)

Efm2302/flu Flu-OO-TCACACAATCGTAAC-NH₂

(Note: Conventional nomenclature used to illustrate the termini of the PNA probe; O=8-amino-3,6-dioxaoctanoic acids; flu=5(6)-carboxy-fluorescein)
Bacterium Strains

Overnight cultures of reference strains (American Type Culture Collection, Manassas, Va.) or clinical isolates representing Enterococcus faecium, other Enterococcus species, including Enterococcus hirae representing the phylogenetically closest related species, and species of other clinical relevant gram-positive cocci were prepared.

Preparation of Smears.

For each strain, smears were prepared on a 8-mm diameter well of a teflon-coated microscope slide (AdvanDx, Woburn, Mass.) by mixing one drop of culture with one drop of phosphate-buffered saline containing 1% (v/v) Triton X-100. The slide was then placed on a 55° C. slide warmer for 20 min at which point the smears were dry. Subsequently, the smears were disinfected by immersion into 96% (v/v) ethanol for 5-10 minutes and air-dried.

Fluorescence In Situ Hybridization (FISH).

Smears were covered with a drop of hybridization solution containing 10% (w/v) dextran sulfate, 10 mM NaCl, 30% (v/v) formamide, 0.1% (w/v) sodium pyrophosphate, 0.2% (w/v) polyvinylpyrrolidone, 0.2% (w/v) ficoll, 5 mM Na₂EDTA, 1% (v/v) Triton X-100, 50 mM Tris/HCl pH 7.5 and 500 nM Efm23S02/flu. Coverslips were placed on the smears to ensure even coverage with hybridization solution, and the slides were subsequently placed on a slide warmer (Slidemoat, Boekel, Germany) and incubated for 90 min at 55° C. Following hybridization, the coverslips were removed by submerging the slides into approximately 20 ml/slide pre-warmed 25 mM Tris, pH 10, 137 mM NaCl, 3 mM KCl in a water bath at 55° C. and washed for 30 min. Each smear was finally mounted using one drop of Mounting medium (AdvanDx, Woburn, Mass.) and covered with a coverslip. Microscopic examination was conducted using a fluorescence microscope equipped with a FITC/Texas Red dual band filter set. Enterococcus faecium was identified by green fluorescent cocci. Results are recorded in Table 2.

TABLE 2

| Organism | Strain ID | Result |
|---|---|---|
| Enterococcus faecium | ATCC 27270 | Positive |
| Enterococcus faecium | ATCC 35667 | Positive |
| Enterococcus faecium | Clinical isolate | Positive |
| Enterococcus faecalis | ATCC 51299 | Negative |
| Enterococcus faecalis | Clinical isolate | Negative |
| Enterococcus gallinarium | ATCC 49573 | Negative |
| Enterococcus hirae | ATCC 8043 | Negative |
| Enterococcus avium | ATCC 14025 | Negative |
| Streptococcus equisimilis | ATCC 12388 | Negative |
| Streptococcus equi | ATCC 9528 | Negative |
| Streptococcus mutans | ATCC 35668 | Negative |
| Streptococcus bovis | ATCC 33317 | Negative |
| Streptococcus agalactiae | ATCC 13813 | Negative |
| Streptococcus pyogenes | ATCC 49399 | Negative |
| Staphylococcus aureus | Clinical isolate | Negative |
| Staphylococcus epidermidis | Clinical isolate | Negative |

An additional experiment using Enterococcus faecalis and Enterococcus faecium as negative and positive control, respectively, was performed where preparation of the smears were performed without the use of one drop of phosphate-buffered saline containing 1% (v/v) Triton X-100 and without immersion of the smears into 96% (v/v) ethanol for 5-10 minutes. This did not affect the results hereby showing that neither treatment with one drop of phosphate-buffered saline containing 1% (v/v) Triton X-100 nor immersion of the smears into 96% (v/v) ethanol for 5-10 minutes are needed for the penetration of the PNA probe through the cell wall during in situ hybridization.

It is concluded that Efm23S02/flu provides rapid and specific identification of Enterococcus faecium by fluorescence in situ hybridization without cross-hybridization to other closely related bacterium species. The assay was performed without the use of cross-linking reagents or enzymes prior to hybridization.

### Example 2

### Analysis of Various Enterococci by Fluorescence In Situ Hybridization

PNA Probe Sequence

(Seq. Id. No.2)

Efm2303/flu Flu-OO- CCTCGAATGTGCTAT-NH₂

(Note: Conventional nomenclature used to illustrate the termini of the PNA probe; O=8-amino-3,6-dioxaoctanoic acids; flu=5(6)-carboxy-fluorescein)
Bacterium Strains

Overnight cultures of reference strains (American Type Culture Collection, Manassas, Va.) or clinical isolates representing Enterococcus faecium, other Enterococcus species, and species of other clinical relevant gram-positive cocci were prepared.

Preparation of Smears.

Smears were prepared as described in Example 1.

Fluorescence In Situ Hybridization (FISH).

Hybridization was performed as described in Example 1 except a different probe, Efm23S03/flu, was used (final probe concentration 250 nM). Microscopic examination was conducted as described above; positively detected cells were identified by green fluorescent cocci. Results are recorded in Table 3.

TABLE 3

| Organism | Result |
|---|---|
| Enterococcus faecium | Positive |
| Enterococcus faecalis | Negative |
| Enterococcus hirae | Positive |
| Enterococcus durans | Positive |
| Enterococcus avium | Negative |
| Streptococcus equisimilis | Negative |
| Streptococcus mutans | Negative |
| Streptococcus agalactiae | Negative |
| Streptococcus pyogenes | Negative |
| Staphylococcus aureus | Negative |
| Staphylococcus epidermidis | Negative |

An additional “No Probe” experiment using hybridization buffer without labeled probe was performed on Enterococcus faecium slides to demonstrate that the cells are not fluorescent in the absence of probe.

With reference to Table 3, it is concluded that Efm23S03/flu provides rapid and specific identification of Enterococcus faecium, Enterococcus durans, and Enterococcus hirae by fluorescence in situ hybridization.

It was noted that the fluorescent signal generated in Enterococcus faecium cells was very bright as compared to Experiment 1. In a follow up experiment, comparison of Efm23S02/flu and Efm23S03/flu used at the same concentration on Enterococcus faecium slides demonstrated that the Efm23S03/flu probe produced a brighter signal.

### Example 3

### Specific Detection of Enterococcus faecium by Fluorescence In Situ Hybridization

PNA Probe Sequences

(Seq. Id. No.2)

| | |
|---|---|
| Efm2303/flu | Flu-OO- CCTCGAATGTGCTAT-NH₂ |

(Seq. Id. No.2Block)

| | |
|---|---|
| Efm2303-B | Ac- CCTCGAATGCGCTAT-NH₂ |

(Note: Conventional nomenclature used to illustrate the termini of the PNA probe; O=8-amino-3,6-dioxaoctanoic acids; flu=5(6)-carboxy-fluorescein; Ac=acetyl cap)
Bacterium Strains

Overnight cultures of reference strains (American Type Culture Collection, Manassas, Va.) representing Enterococcus faecium, other Enterococcus species were prepared.

Preparation of Smears.

Smears were prepared as described in Example 1.

Fluorescence In Situ Hybridization (FISH).

Hybridization was performed as described in Example 2 except a second probe, Efm23S03-B, was also added at 250 nM. Efm23S03-B is a non-labeled blocker probe which differs from Efm23S03/flu by one base. The one base makes the Efm23S₀₃-B probe specific to E. durans and E. hirae. Microscopic examination was conducted as described above; positively detected cells were identified by green fluorescent cocci. Results are recorded in Table 4.

TABLE 4

| Organism | Result |
|---|---|
| Enterococcus faecium | Positive |
| Enterococcus faecalis | Negative |
| Enterococcus hirae | Negative |
| Enterococcus durans | Negative |

With reference to Table 4, it is concluded that inclusion of Efm23S03-B in the hybridization with Efm23S03/flu allows specific identification of Enterococcus faecium by fluorescence in situ hybridization without detection of Enterococcus durans, and Enterococcus hirae.

### Example 4

### Multicolor Detection of Various Enterococci by Fluorescence In Situ Hybridization

PNA Probe Sequences

(Seq. Id. No.2)

| | |
|---|---|
| Efm2303/flu | Flu-OO- CCTCGAATGTGCTAT-NH₂ |

(Seq. Id. No.2Block)

| | |
|---|---|
| Efm2303-B | Ac- CCTCGAATGCGCTAT-NH₂ |
| Efs16S01/flu | Flu-OO- CCTCTGATGGGTAGG -NH₂ |
| Efm16S01/tam | Tam-OO- CCTTCTGATGGGCAG-NH₂ |

(Note: Conventional nomenclature used to illustrate the termini of the PNA probe; O=8-amino-3,6-dioxaoctanoic acids; flu=5(6)-carboxy-fluorescein; Ac=acetyl cap, tam=5(6)-carboxytetramethyrhodamine)
Bacterium Strains

Overnight cultures of reference strains (American Type Culture Collection, Manassas, Va.) representing Enterococcus faecium, other Enterococcus species were prepared.

Preparation of Smears.

Smears were prepared as described in Example 1.

Fluorescence In Situ Hybridization (FISH).

Hybridization was performed as described in Example 3 except Efm23S03-B, was added at 500 nM, and Efs16S01/flu and Efm16S01/tam were both added at 250 nM. Microscopic examination was conducted as described above; positively detected cells were identified by fluorescent cocci. Results are recorded in Table 5.

TABLE 5

| Organism | Result/Color |
|---|---|
| Enterococcus faecium | Positive/Gold |
| Enterococcus faecalis | Positive/Green |
| Enterococcus hirae | Positive/Red |
| Enterococcus durans | Positive/Red |

With reference to Table 5, each species tested was detected by red, green or gold fluorescence. E. faecalis was detected with the Efs16S01/flu probe resulting in green fluorescence. E. durans and E. hirae were detected with the Efm16S01/tam probe resulting in red fluorescence. The presence of the blocker probe, Efm23S03-B, prevented secondary detection of E. durans and E. hirae by the Efm23S03/flu probe. E. faecium was detected by both the Efm23S03/flu and Efm16S01/tam probes resulting in the golden color, a mixture of red and green fluorescence from the Tam and Flu labels respectively.

Equivalents

While this invention has been particularly shown and described with references to preferred embodiments thereof, it will be understood by those of ordinary skill in the art that various changes in form and details may be made therein without departing from the spirit and scope of the invention as defined by the appended claims. Those skilled in the art will be able to ascertain, using no more than routine experimentation, many equivalents to the specific embodiments of the invention described herein. Such equivalents are intended to be encompassed in the scope of the claims.

The disclosures of all references mentioned herein are incorporated by reference.

1. A PNA probe comprising a nucleobase sequence suitable for the analysis of Enterococcus faecium, said PNA probe being complementary to a target sequence of Enterococcus faecium 23 rRNA or rDNA or its complement.

2. A PNA probe of claim 1 comprising a nucleobase sequence suitable for the detection, identification and/or quantitation of Enterococcus faecium, said PNA probe being complementary to a target sequence of Enterococcus faecium 23 rRNA or rDNA or its complement.

3. A PNA probe of claim 1 comprising a nucleobase sequence suitable for the determination of antibiotic resistance of Enterococcus faecium, said PNA probe being complementary to a target sequence of Enterococcus faecium 23 rRNA or rDNA or its complement.

4. The PNA probe of claim 1-3, wherein at least a portion of the probe is at least about 86% identical to the nucleobase sequence or complement thereof selected from the following sequences: TCA CAC MT CGT MC (Seq. Id. No. 1), CCT CGA ATG TGC TAT (Seq. Id. No. 2), CCC AGC TAG CGT GCC (Seq. Id. No. 3) and CCC AGC TCG CGT GCC (Seq. Id. No. 4) or the complements.

5. The PNA probe of claim 4, wherein the probe sequence is 8-17 subunits in length.

6. The PNA probe of claim 1 for the detection, identification and/or quantification of Enterococcus faecium selected from the following probe sequences: TCA CAC MT CGT AAC (Seq. Id. No. 1) and CCT CGA ATG TGC TAT (Seq. Id. No. 2) or the complements.

7. The PNA probe of claim 1 for the determination of antibiotic resistance of Enterococcus faecium selected from the following probe sequences: CCC AGC TAG CGT GCC (Seq. Id. No. 3) and CCC AGC TCG CGT GCC (Seq. Id. No. 4) or the complements.

8. The PNA probe of claim 1 wherein the probe is labeled with at least one detectable moiety.

9. The PNA probe of claim 8, wherein the detectable moiety or moieties are selected from the group consisting of: a conjugate, a branched detection system, a chromophore, a fluorophore, a spin label, a radioisotope, an enzyme, a hapten, an acridinium ester and a luminescent compound.

10. The PNA probe of claims 1-9, wherein the probe is self-reporting.

11. The PNA probe of claim 10, wherein the probe is a PNA Linear Beacon.

12. The PNA probe of claim 1, wherein the probe is unlabeled.

13. The PNA probe of claim 1, wherein the probe is bound to a support.

14. The PNA probe of claim 13, wherein the probe further comprises a spacer or a linker.

15. The PNA probe of claim 1, wherein in situ hybridization is used for analysis of Enterococcus faecium optionally present in the sample.

16. The PNA probe set of claim 1, wherein two or more PNA probe are used for analysis of Enterococcus faecium.

17. The PNA probe set of claim 1, wherein the probes are differently labeled for independent analysis.

18. The PNA probe set of claim 1 selected from the following nucleobase sequences: TCA CAC AAT CGT AAC (Seq. Id. No. 1) and CCT CGA ATG TGC TAT (Seq. Id. No. 2) or the complements thereof together with corresponding blocker probes selected from the following nucleobase sequences TCA CGC AAA CGT AAC (Seq. Id No. 5) and CCT CGA ATG CGC TAT (Seq. Id. No. 6) or the complements thereof.

19. A method for the detection, identification and/or quantitation of Enterococcus faecium in a sample, said method comprising:
a) contacting at least one of the PNA probes of claims 1-18 to the sample,
b) hybridizing the PNA probe to a target sequence of Enterococcus faecium in the sample; and
c) detecting the hybridization as being indicative of presence, identity and/or amount of Enterococcus faecium in the sample.

20. A method according to claim 19, wherein the analysis takes place in situ.

21. A method according to claim 20, wherein the analysis takes place by fluorescence in situ hybridization.

22. A method according to claim 19, wherein the method does not involve the use of cross-linking reagents or enzymes prior to hybridization.

23. The method of claim 19, wherein the method is used to detect a nucleic acid comprising a target sequence wherein said nucleic acid has been synthesized or amplified in a reaction.

24. The method of claim 23 wherein preferred nucleic acid synthesis or nucleic acid amplification reactions are selected from the group consisting of: Polymerase Chain Reaction (PCR), Ligase Chain Reaction (LCR), Strand Displacement Amplification (SDA), Transcription-Mediated Amplification (TMA), Rolling Circle Amplification (RCA) and Q beta replicase.

25. The method of claim 19, wherein the method further comprises adding at least one blocking probe to reduce or eliminate any hybridization of the PNA probe to non-target sequence.

26. The method of claim 25, wherein the target sequence is immobilized to a surface.

27. The method of claim 19, wherein said PNA probe is immobilized to a surface.

28. The method of claim 27, wherein said PNA probe is one component of an array.

29. The method of claim 19, wherein the method comprises the use of a PNA probe set of claims 16-18.

30. The method of claim 19, wherein the sample is a biological sample.

31. The method of claim 30, wherein the biological sample is blood, urine, secretion, sweat, sputum, stool, mucous, or cultures thereof.

32. A kit suitable for performing an assay for analysis of Enterococcus faecium in a sample, wherein said kit comprises: a) a PNA probe according to claim 1 and b) other reagents or compositions necessary to perform the assay.

33. The kit of claim 32, wherein Enterococcus faecium and at least one other microorganism optionally present in a sample are independently detected, identified and/or quantitated.

34. The kit of claim 32, wherein Enterococcus faecium optionally present in a sample is detected, identified and/or quantitated and its susceptibility to antimicrobial agents is determined.

35. The kit of claim 32, wherein the kit is used in an in-situ hybridization assay.

36. The kit of claim 32, wherein the kit is used for a real-time PCR assay.

37. The kit of claim 32, wherein the kit is used to examine clinical samples such as clinical specimens or cultures thereof.

38. A method for treating of patient, comprising
a) obtaining a biological sample from the patient
b) determining the presence, amount and/or identity of E. faecium; and
c) administering at least one antibiotic with activity towards vancomycin resistant enterococci (VRE).

39. The method of claim 38, where the antibiotic is linezolid, quinupristindalfopristin, or daptomycin.

40. The method of claim 38, wherein the patient has or is suspected of harboring a vancomycin resistant enterococci (VRE).

41. The PNA probe set comprising at least the PNA probe of claim 1, wherein two or more probes are used to create a third color by coincidental fluorescence.

42. The PNA probe set of claim 41, wherein the PNA probes comprise CCTCGAATGTGCTAT (Seq. Id. No. 2), CCTCGAATGCGCTAT (Seq. Id. No. 6), CCTCTGATGGGTAGG (SEQ ID NO: 7) and CCTTCTGATGGGCAG (SEQ ID NO: 8).
